(19) United States
(12) Patent Application Publication
(10) Pub. No.: US 2017/0087120 A1
(43) Pub. Date: Mar. 30, 2017

(54) COMPOSITION FOR IMPROVING BIOAVAILBILITY AND EFFICACY OF TAXANE

(71) Applicant: Florida A&M University, Tallahassee, FL (US)

(72) Inventors: Mandip Singh Sachdeva, Tallahassee, FL (US); Ketankumar Patel, Tallahassee, FL (US); Chandraiah Godugu, Khammam Dist (IN)

(21) Appl. No.: 15/248,452

(22) Filed: Aug. 26, 2016

Related U.S. Application Data
(60) Provisional application No. 62/210,206, filed on Aug. 26, 2015.

(51) Int. Cl. A61K 31/337; A61K 31/4412

(52) U.S. Cl. CPC A61K 31/337; A61K 31/4412

(57) ABSTRACT

In an embodiment, the current invention is a composition for oral or parenteral administrable taxane to treat cancer in human patients, comprising orally or parenterally co-administering to said patient a taxane comprising a derivative, an analog or a prodrug of paclitaxel or docetaxel, and bioavailability and efficacy enhancing agent comprising a piperlongumine. The present invention relates in its principal aspect to the oral or parenteral administration of one or a combination of taxanes to human patients suffering from cancer. In another embodiment, the current invention is a method of increasing the bioavailability of taxanes, which are poorly absorbed or not absorbed at all from the gastrointestinal tract or gut, by pre-administering and/or simultaneously administering to a human subject orally or parenterally one or a combination of agents (“enhancing agents”) effective in enhancing bioavailability by inhibiting CYP3A4 enzyme. In yet another embodiment, the current invention is a method for enhancing the cytotoxicity of taxanes with synergistic interaction in variety to cancer cells.

### CROSS-REFERENCE TO RELATED APPLICATIONS

This nonprovisional application claims priority to U.S. Provisional Patent Application No. 62/210,206, entitled “Composition for Improving Bioavailbility and Efficacy of Taxane”, filed Aug. 26, 2015 by the same inventors, the entirety of which is incorporated herein by reference.

### BACKGROUND OF THE INVENTION

1. Field of the Invention

This invention relates, generally, to taxanes. More specifically, it relates to compositions that improve the bioavailability and efficacy of taxanes.

2. Brief Description of the Prior Art

Very low oral bioavailability due to extensive pre-systemic metabolism and P-glycoprotein (P-gp) efflux has constrained the oral metronomic chemotherapy of taxanes including paclitaxel and docetaxel. Further, dose dependent side effects and development of drug resistance adversely affecting parenteral chemotherapy by taxane, taxane analogs or taxane derivatives, e.g., paclitaxel, docetaxel, or cabazitaxel.

Attempts have been made to increase bioavailability and efficacy of taxanes. Examples include PCT Application No. PCT/GB2008/002854; U.S. Pat. No. 6,730,698; Park J H et al., Effects of silymarin and formulation on the oral bioavailability of paclitaxel in rat, Eur J Pharm Sci, 45(3):296-301, 2012 Feb. 14; Malingre M M et al., Coadministration of cyclosporine strongly enhances the oral bioavailability of docetaxel, Journal of clinical oncology: official journal of the American Society of Clinical Oncology, 19(4):1160-1166, 2001; Ganta S et al., Curcumin enhances oral bioavailability and anti-tumor therapeutic efficacy of paclitaxel upon administration in nanoemulsion formulation, Journal of pharmaceutical sciences, 99(11):4630-4641, 2010; Choi Y H et al., Effects of tesmilifene, a substrate of CYP3A and an inhibitor of P-glycoprotein, on the Pharmacokinetics of intravenous and oral docetaxel in rats, The Journal of pharmacy and pharmacology, 62(8):1084-1088, 2010; and Gong L H et al., Piperlongumine induces apoptosis and synergizes with cisplatin or paclitaxel in human ovarian cancer cells, Oxidative medicine and cellular longevity, 2014:906804. However, none have been completely effective in solving oral bioavailability of taxanes.

Accordingly, what is needed is a compound that facilitates the oral delivery of docetaxel and improves the efficacy of docetaxel. However, in view of the art considered as a whole at the time the present invention was made, it was not obvious to those of ordinary skill in the field of this invention how the shortcomings of the prior art could be overcome.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF DESCRIPTION OF THE DRAWINGS

For a fuller understanding of the invention, reference should be made to the following detailed description, taken in connection with the accompanying drawings, in which:

FIGS. 1A-1C are graphical illustrations depicting the effect of piperlongumine (PPL) on docetaxel (DTX) metabolism, efflux, and oral absorption. In FIG. 1A, in vitro microsomal metabolism studies of DTX by human liver microsomes were carried out in combination with PPL and compared with a known standard CYP3A4 inhibitor, Cyclosporin A (CYA). Addition of PPL in reaction mixtures significantly reduced the metabolism of DTX and doubled the half-life of DTX. PPL showed CYP3A4 inhibition comparable to inhibitor CYA. In FIG. 1B, Caco-2 is colon carcinoma cells used to evaluate the permeability and efflux of drug in GI track. DTX has efflux ratio above 2 which indicate that it is a substrate for efflux transporter. Effect of DTX permeability across caco-2 monolayer was evaluated in the presence of PPL and a known standard P-gp efflux inhibitor, Verapamil. PPL showed marked reduction in DTX efflux ratio comparable to standard agent. In FIG. 1C, in vivo oral pharmacokinetic of DTX alone and coadministration with PPL was carried out in SD rats. PPL led to significant enhancement in DTX oral bioavailability.

FIG. 2A depicts cytotoxicity of DTX and DTX-PPL combination of various cell lines using crystal violet assay.

FIG. 2B depicts significant alteration in apoptosis markers observed in DTX-PPL treatment compared to both the drugs alone.

FIG. 2C depicts in vitro migration assay on MDA-MB-231 cell line. PPL showed significant reduction in % bridging of migration area.

### DETAILED DESCRIPTION OF THE PREFERRED EMBODIMENT

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

As used in this specification and the appended claims, the singular forms “a”, “an”, and “the” include plural referents unless the content clearly dictates otherwise. As used in this specification and the appended claims, the term “or” is generally employed in its sense including “and/or” unless the context clearly dictates otherwise.

In an embodiment, the current invention is a composition for oral or parenteral administrable taxane to treat cancer in human patients, comprising orally or parenterally co-administering to said patient a taxane comprising a derivative, an analog or a prodrug of paclitaxel or docetaxel, and bioavailability and efficacy enhancing agent comprising a piperlongumine. The present invention relates in its principal aspect to the oral or parenteral administration of one or a combination of taxanes to human patients suffering from cancer.

In another embodiment, the current invention is a method of increasing the bioavailability of taxanes, which are poorly absorbed or not absorbed at all from the gastrointestinal tract or gut, by pre-administering and/or simultaneously administering to a human subject orally or parenterally one or a combination of agents (“enhancing agents”) effective in enhancing bioavailability by inhibiting CYP3A4 enzyme.

Piperlongumine showed reduction in paclitaxel and docetaxel metabolism by human microsomal enzyme, more specifically, CYP3A4 enzymes. Reduction in efflux ratio and enhancement in permeability co-efficient of paclitaxel and docetaxel across the Caco-2 monolayer were observed when co-treated with piperlongumine. Piperlongumine has been seen to significantly enhance the oral bioavailability of docetaxel in SD rats.

In another embodiment, the current invention is a method for enhancing the cytotoxicity of taxanes with synergistic interaction in variety to cancer cells, including lung cancer, breast cancer, colon cancer, pancreatic cancer, kidney cancer, brain cancer, triple negative breast cancer, liver cancer, etc. The IC50 value of paclitaxel and docetaxel was reduced 3-5 times and combination index values for cytotoxicity in all the cell lines were below 0.6. Expression of tumor markers such as survivin, bcl2, C-myc and cyclin D1 were down regulated to a great extent with enhanced p53 expression when treated with combination instead of individual drug.

Piperlongumine has also enhanced the anti-migration effect of docetaxel and paclitaxel in cancer cell lines. Use of piperlongumine with taxane offers various advantages, for example including, but not limited to, the following:
1. Enhance the oral and intravenous bioavailability of taxanes including docetaxel and paclitaxel.
2. Enhance the cytotoxicity of docetaxel and paclitaxel on various cancer cell lines including lung cancer, breast cancer, colon cancer, pancreatic cancer, liver cancer, triple negative breast cancer, brain cancer, head and neck cancer etc.
3. Reduce the docetaxel or paclitaxel induced side effects/toxicities.
4. Can help in reducing the dose of docetaxel or paclitaxel.

PCT Patent Application No. PCT/GB2008/002854 discusses pharmaceutical compositions and methods for the treatment of neoplastic disease and comprising the combination of a taxane, such as docetaxel, with a CYP3A4 inhibitor, such as ritonavir. U.S. Pat. No. 6,730,698 discusses a method and compositions for administering taxanes orally to human patients. It describes the use of oral cyclosporine A for enhancing oral bioavailability of paclitaxel. However, these are not completely effective, in particular in view of studies of the current invention. In light of the foregoing references, salient features/advantages of certain embodiments of the current invention may include, but are not limited to, the following:
1. Piperlongumine enhances the oral and parenteral bioavailability of taxanes, thus permitting more taxane to be available to the tumor.
2. Piperlongumine inhibits cyp3a4 enzyme and P-gp efflux pump, which are the major limiting factor for oral absorption of taxanes.
3. Piperlongumine enhances the cytotoxicity effect of taxane, so higher anticancer efficacy can be achieved with lower doses of taxanes, which is very helpful in minimizing any side effects of the therapy.
4. Oral metronomic chemotherapy with taxanes including docetaxel and paclitaxel is possible if we deliver it with piperlongumine.

### EXAMPLE

In an embodiment, the current invention is a composition for oral or parenteral administrable taxane to treat cancer in human patients, comprising orally or parenterally co-administering to said patient a taxane comprising a derivative, an analog or a prodrug of paclitaxel or docetaxel, and bioavailability and efficacy enhancing agent comprising a piperlongumine.

The present invention relates in its principal aspect to the oral or parenteral administration of one or a combination of taxanes to human patients suffering cancer. A preferred embodiment of the invention is a method of increasing the bioavailability of taxanes, which are poorly absorbed or not absorbed at all from the gastrointestinal tract or gut, by pre-administering and/or simultaneously administering to a human subject by the oral or parenteral route one or a combination of agents (“enhancing agents”) effective in enhancing bioavailability by inhibiting CYP3A4 enzyme. Very low oral bioavailability due to extensive pre-systemic metabolism and P-gp efflux has constrained the oral metronomic chemotherapy of taxanes while dose dependent side effects and development of drug resistance adversely affecting parenteral chemotherapy by taxane, taxane analogs or taxane derivatives (e.g., paclitaxel, docetaxel or cabazitaxel).

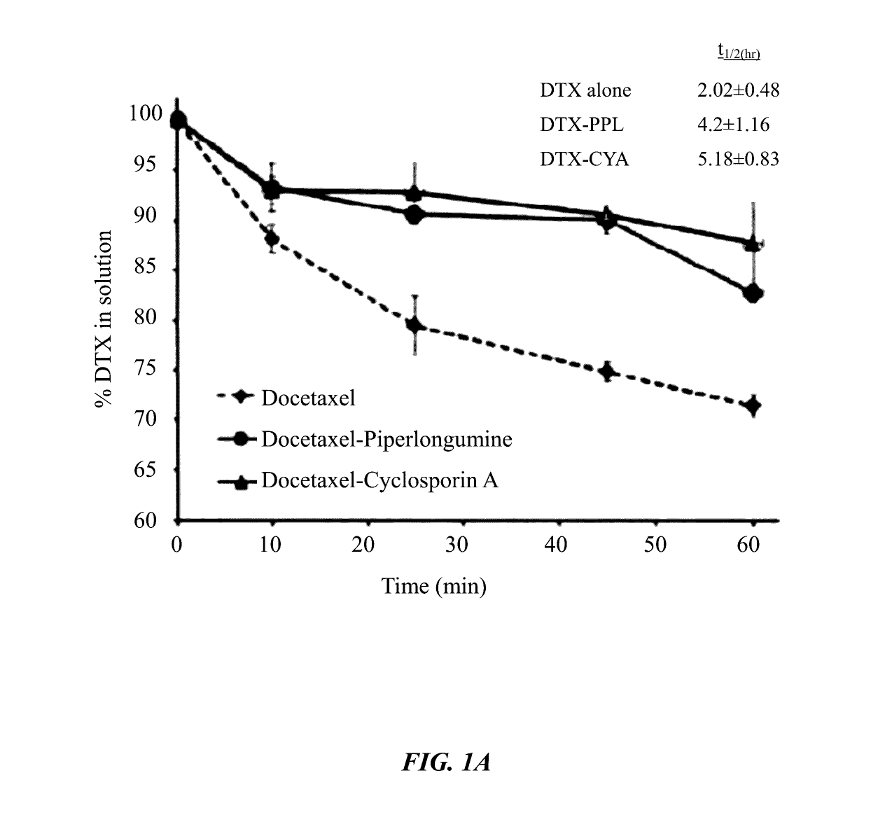

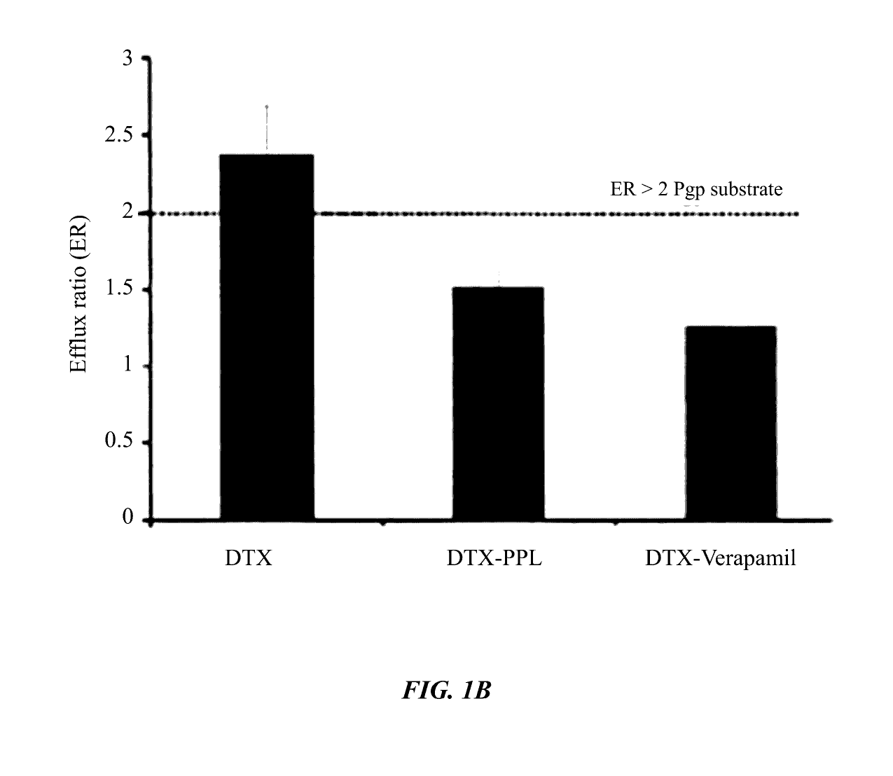

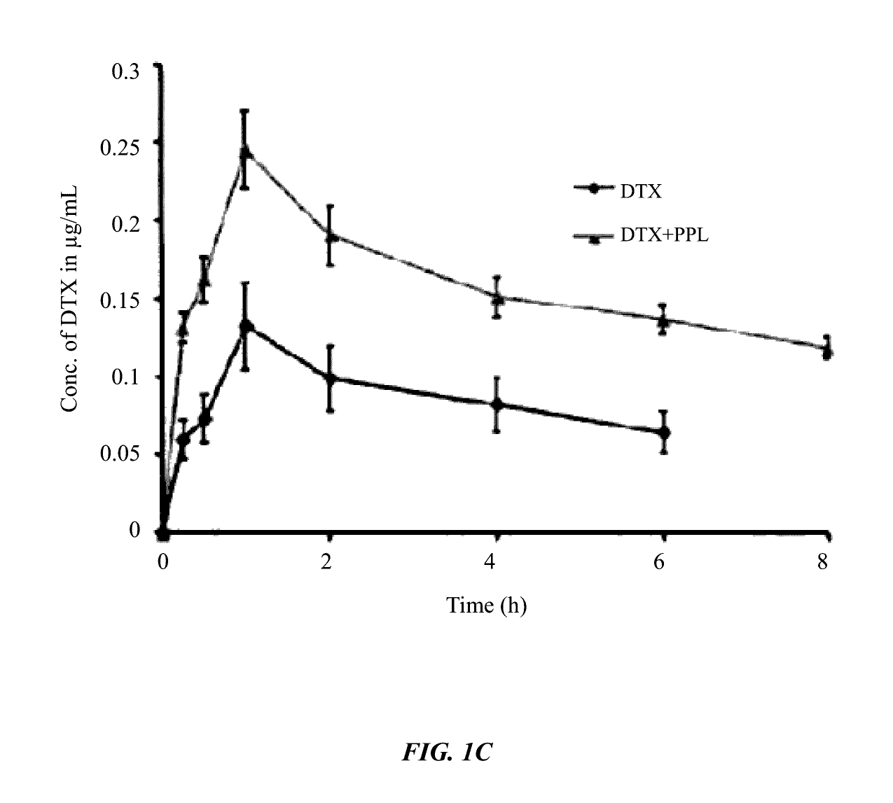

Piperlongumine showed reduction paclitaxel and docetaxel metabolism by human microsomal enzyme, more specifically, CYP3A4 enzymes, as seen in FIG. 1A. Reduction in efflux ratio and enhancement in permeability co-efficient of paclitaxel and docetaxel across Caco-2 monolayer were observed when co-treated with piperlongumine (FIG. 1B). Piperlongumine significantly enhanced the oral bioavailability of docetaxel in SD rats, as seen in FIG. 1C.

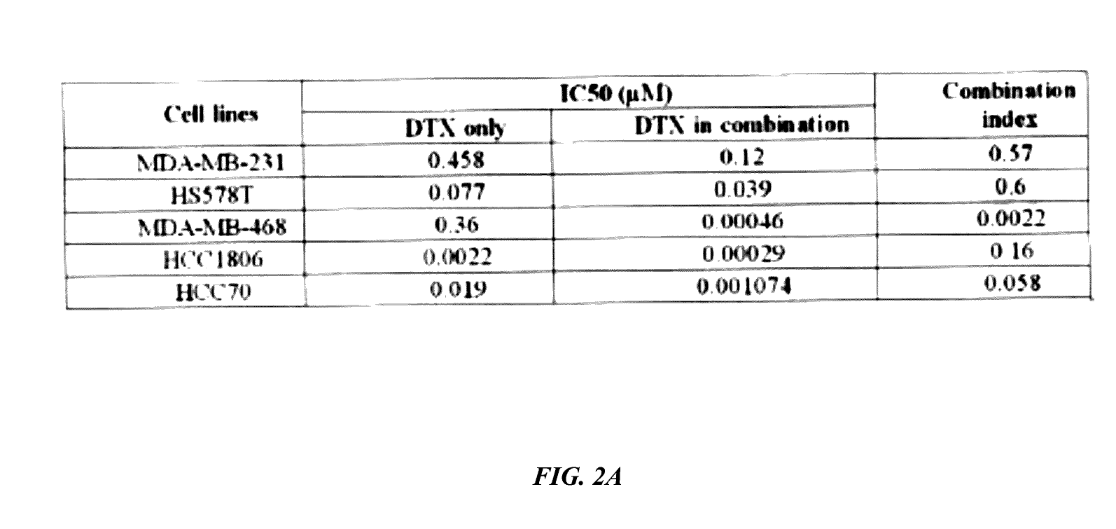

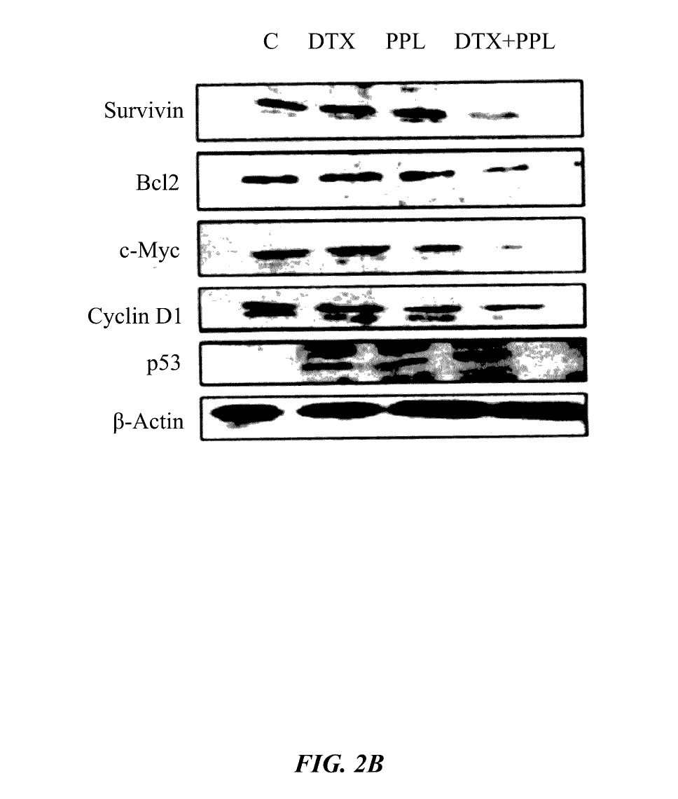

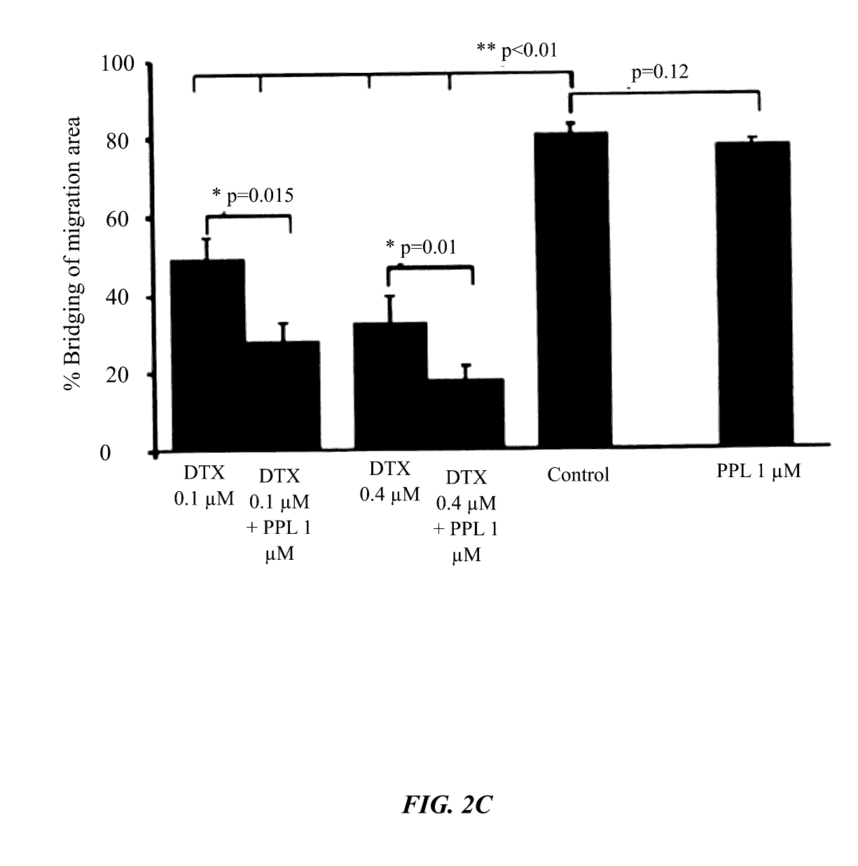

In another embodiment, the current invention is a method for enhancing the cytotoxicity of taxanes with synergistic interaction in variety to cancer cells including lung cancer, breast cancer, colon cancer, pancreatic cancer, kidney cancer, brain cancer, triple negative breast cancer, liver cancer, etc. The IC50 value of paclitaxel and docetaxel was reduced 3-5 times and combination index values for cytotoxicity in all the cell lines were below 0.6 (FIG. 2A).

Expression of tumor markers such as survivin, bcl2, C-myc and cyclin D1 were down regulated to a great extent with enhanced p53 expression when treated with combination instead of individual drug (FIG. 2B). Piperlongumine has also enhanced the anti-migration effect of docetaxel and paclitaxel in cancer cell lines (FIG. 2C).

### DEFINITIONS

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients, in the specified amounts, as well as any product which results, directly or indirectly, from combination of the specified ingredients in the specified amounts. Generally, the specified ingredients, or pharmaceutically acceptable salts and derivatives thereof, are suitable agents for use in the diagnosis, mitigation, treatment, cure, or prevention of disease in a subject, specifically but not exclusively effective in the treatment and/or prevention of cancers, when administered in an effective amount to a subject in need thereof.

As used herein, the term “neoproliferative disease” means a neoplasm, cancer, or precancerous lesion. The neoplasm or cancer may be benign or malignant.

As used herein, “patient”, “subject” and “subject in need of treatment” are used interchangeably to mean mammals in need of diagnosis or treatment for a cancer or pre-cancer or tumor thereof.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers, such as a solvent, suspending agent or vehicle, for delivering the compound or compounds in question to the mammal. The carrier may be liquid or solid and is selected with the planned manner of administration in mind. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, liposomes, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. The carrier can also include any and all other vehicles, coatings, diluents, antibacterial and antifungal agents, isotonic and absorption delaying agents, buffers, carrier solutions, suspensions, colloids, and the like. Formulations are described in a number of sources that are well known and readily available to those skilled in the art. Except insofar as any conventional media or agent is incompatible with the active ingredient, its use in the therapeutic compositions is contemplated. For example, Remington's Pharmaceutical Sciences (Martin E W [1995] Easton Pa., Mack Publishing Company, 19^{th} ed.) describes formulations which can be used in connection with the subject invention.

Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question. The pharmaceutical composition can be adapted for various forms of administration. Administration can be continuous or at distinct intervals as can be determined by a person skilled in the art.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

As used herein, the term “precancerous” refers to cells or tissues that have characteristics relating to changes that may lead to malignancy or cancer, such as mutations controlling cell growth and proliferation. Examples include adenomatous growths in breast and prostate tissue, or for example, conditions of dysplastic nevus syndromes, polyposis syndromes, prostatic dysplasia, and other neoplasms, whether clinically identifiable or not.

The term “prevention” is used herein to refer to the management of the factors that could lead to disease or disorder, so as to prevent the occurrence of the disease or disorder. Specifically, the disease or disorder includes, but is not limited to, cancer.

A “safe and effective amount” refers to the quantity of a component or composition that is sufficient to yield a desired therapeutic response without undue adverse side effects (such as toxicity, irritation, or allergic response) commensurate with a reasonable benefit/risk ratio when used in the manner of this invention for the treatment and/or prevention of cancer in a subject in need thereof.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more doses. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

As used herein, “treatment” refers to obtaining beneficial or desired clinical results, or any measurable mitigation of disease in a subject, including resolution, reduction, halting progression, and/or slowing progression of a disease. Beneficial or desired clinical results include, but are not limited to, any one or more of the following: alleviation of one or more symptoms (such as tumor growth or metastasis), diminishment of extent of cancer, stabilized (i.e., not worsening) state of cancer, preventing or delaying spread (e.g., metastasis) of the cancer, preventing or delaying occurrence or recurrence of cancer, delay or slowing of cancer progression, amelioration of the cancer state, remission (whether partial or total). The methods of the invention contemplate any one or more of these aspects of treatment.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A composition for oral or parenteral administrable taxane to treat cancer in human patients, comprising orally or parenterally co-administering to said patient a taxane comprising a derivative, an analog, or a prodrug of paclitaxel or docetaxel, and bioavailability and efficacy enhancing agent comprising a piperlongumine.

2. An oral or parenteral administrable composition for treating cancer in a patient, comprising:
a therapeutically effective amount of taxane including a derivative, an analog, or a prodrug of paclitaxel or docetaxel; and
a therapeutically effective amount of a bioavailability and efficacy enhancing agent including a piperlongumine,
wherein said taxane and said piperlongumine are co-administered to said patient.

3. A method of increasing the bioavailability of taxanes, comprising pre-administering or simultaneously administering to a human subject orally or parenterally one or a combination of enhancing agents effective in enhancing bioavailability by inhibiting CYP3A4 enzyme.
